(19) United States
(12) Patent Application Publication
(10) Pub. No.: US 2009/0202586 A1
(43) Pub. Date: Aug. 13, 2009

(54) ATTENUATED VACCINE FOR TULAREMIA

(75) Inventors: Burt Anderson, Valrico, FL (US); Wendy Sammons, Rockville, MD (US); Jean Citron, Seminole, FL (US); Chandra Shekhar Bakshi, Glenmont, NY (US); Dennis Metzger, Niskayuna, NY (US)

Correspondence Address: SMITH HOPEN, PA, 180 PINE AVENUE NORTH, OLDSMAR, FL, 34677, US

(73) Assignees: UNIVERSITY OF SOUTH FLORIDA, Tampa, FL (US); ALBANY MEDICAL COLLEGE, Albany, NY (US); BAY PINES VA HEALTHCARE SYSTEM, Bay Pines, FL (US)

(21) Appl. No.: 12/016,567

(22) Filed: Jan. 18, 2008

Related U.S. Application Data
(60) Provisional application No. 60/885,556, filed on Jan. 18, 2007.

(51) Int. Cl. A61K 39/02; C12N 1/21; C12N 15/11

(52) U.S. Cl. 424/200.1; 435/252.3; 536/23.7

(57) ABSTRACT

A scan of F. tularensis genome for homology to a regulatory protein that controls virulence identified gene FTL0552. A knock out mutation in FTL0552 was created using reverse transcriptase PCR and the construct inserted into F. tularensis. This mutant was defective for survival in macrophages and found avirulent in in vivo testing, where the mutant exhibited reduced levels of pro-inflammatory cytokine production, reduced evidence of histopathology in affected tissues, reduced systemic infection, and rapid clearance of the bacterium. In vivo challenge studies with the FTL0552 mutant using the virulent F. tularensis subsp. tularensis SchuS4 strain show an immune response is induced, and protection afforded, after preexposure to the FTL0552 mutant. Microarray studies revealed 148 genes regulated by FTL0552, including genes located within the FPI that are essential for intracellular survival.

### CROSS REFERENCE TO RELATED APPLICATION

This application claims priority to currently pending U.S. Provisional Patent Application No. 60/885,556, entitled “Methods for the Development of an Attenuated Vaccine Candidate Strain of Francisella Tularensis”, filed on Jan. 18, 2007, the contents of which are herein incorporated by reference.

### FIELD OF INVENTION

This invention relates to vaccines. Specifically, the invention relates to vaccines for tularemia based on Francisella tularensis.

### BACKGROUND OF THE INVENTION

Francisella tularensis is a small, non-motile, aerobic, gram-negative cocco-bacillus, the only genus belonging to the Family Francisellaceae and a member of the γ-subclass of proteobacteria. This bacterium was first discovered following an outbreak of a plague-like illness in ground squirrels in Tulare County, California. The bacterium is a hardy, non-spore forming organism, with a thin lipopolysaccharide-containing envelope that can persist in the environment for long periods of time in low temperature water, moist soil, hay, straw, and decaying animal carcasses. There are five subspecies of F. tularensis found in the Northern Hemisphere where two, subspecies tularensis and subspecies holartica, cause human disease. The most virulent subspecies, tularensis (type A), is the causative agent of the zoonotic disease, tularemia. It is predominantly found in North America and is associated with lethal pulmonary infections. Recently, the tularensis subspecies has been divided into genetically distinct type A1 and A2. The second subspecies, holartica (type B), is found mostly in Europe and Asia and rarely causes a fatal disease in humans.

An attenuated Live Vaccine Strain (LVS), derived from holartica, has been described and shown to offer protection to humans against naturally and laboratory acquired tularemia but remains as virulent to mice as wild type subspecies holartica. LVS causes disease in mice that is virtually indistinguishable from that caused in humans by highly virulent strains. The LVS strain is not fully licensed as a vaccine but is currently under review by the U.S. Food and Drug Administration.

F. tularensis subsp. tularensis has been classified by the United States Centers for Disease Control and Prevention (CDC) Strategic Planning Group as a Category A agent of high priority, due to its virulence, low infective dose, and its potential for transmission by aerosol. In humans, an infectious dose for type A strains can be as low as 10 bacteria for respiratory or intradermal routes.

Little is known about virulence mechanisms of F. tularensis. Macrophages are believed to be the primary host cell for survival and replication of the bacterium, where the ability of Francisella species to survive and multiply in macrophages plays a crucial role in its pathogenesis. However, the exact niche occupied by this organism and virulence factors modulating the organism's intracellular growth and survival are not clearly defined. Recent studies identified a 23-kDa protein, encoded by the intracellular growth locus (iglC) whose expression is upregulated in macrophages. The iglC gene is located within the iglABCD gene cluster that is a major component of the FPI. The expression of FPI genes required for intramacrophage survival, including iglB and iglC, is controlled by the macrophage growth locus A and B (MglA and MglB). MglA and MglB are transcriptional regulators controlling the expression of virulence genes. Mutants in mglA, mglB, iglB, and mglC do not escape the phagosome, have impaired intracellular macrophage growth, and dramatically reduced virulence in a mouse model.

The pathogen-host relationship is complex, and successful infection depends upon the expression of a number of bacterial genes adapted for infection of the host. In the case of the vector-borne zoonotic bacterium F. tularensis, it must survive within arthropod vectors, such as ticks, as well as in warm-blooded vertebrate hosts. The synthesis of virulence factors in these environments is highly regulated and responds to environmental cues such as growth phase, temperature, osmotic stress, and changing concentration of extracellular ions such as Mg²⁺, Ca²⁺, and Fe²⁺. Two component signal transduction systems (TCS) are the most prevalent strategies bacteria use to couple environmental signals to adaptive responses, and play an important role in bacterial survival under environmental stress including survival within macrophages. They typically contain a membrane bound sensor kinase and a cytoplasmic response-regulator. The sensor kinase detects environmental signals received at the surface of the cells, resulting in autophosphorylation of a histidine residue in the cytoplasmic C-terminal tail.

The response-regulator is comprised of two highly conserved domains, the regulatory/receiver domain and the effector domain. Inactivation of two component signal transduction systems results in reduced bacterial virulence. In Salmonella phoP/phoQ genes control the expression of more than 40 genes, important for intramacrophage survival during infection. The PhoQ sensor kinase responds to changes in the external environment such as magnesium concentrations to activate the PhoP response-regulator, and results in the transcription of genes essential for survival of the bacteria in the changing environment. PhoP also controls virulence in Yersinia pestis, Shingella flexneri, Myocobacterium tuberculosis, Bordetella pertussis, and N. meningitides.

### SUMMARY OF THE INVENTION

PmrA, a TCS response-regulator, was recently described in Francisella tularensis subsp. novicida. PmrA is an orphan member of the typical two-component regulatory systems, and shares high similarity (44%) with the Salmonella PmrA response-regulator. F. novicida mutants lacking the pmrA gene had reduced survival and growth within macrophages and offer complete protection in mice against homologous challenge but did not protect the mice against challenge with the SchuS4 strain.

Locus FTL0552 was identified, through protein product homology, to the PhoP response-regulator consensus sequence. FTL0552 is annotated in the genomic sequence as a transcriptional response regulator. The corresponding locus in F. tularensis SchuS4 (FTT1 557c) is highly conserved.

### BRIEF DESCRIPTION OF THE DRAWINGS

For a fuller understanding of the invention, reference should be made to the following detailed description, taken in connection with the accompanying drawings, in which:

FIG. 1 is a diagram of the arrangement of F7L0552 and adjacent genes on the F. tularensis LVS genome (NC007880). The genome locus tags are indicated as are the size of the genes in base pairs (top) and the intergenic space between genes (below). The genes indicated are: Ψ; pseudogene; putative response-regulator, lepB; signal peptidase I, rnc; RNase III, truB; tRNA pseudouridine synthetase B, rnr; RNaseR, and olel; delta9 acyl-lipid fatty acid desaturase. The gene arrangement is suggestive of a five-gene operon transcribed from a promoter region upstream of FTL0552.

FIG. 2 is a table of the DNA microarray analysis of genes identified by at least a four-fold difference between F. tularensis LVS parent strain and FTL0552 mutant. Results are based on the change between five independent F. tularensis LVS parent strain and FTL0552 mutant microarrays.

FIG. 3 is a table of the construct primers for the knock out mutation and adaptors.

FIG. 4 is a diagram of the knock out construct and flanking regions. The regions flanking FTL0552 are amplified separately by PCR and ligated to a formed PCR construct containing kanamycin resistance. The ligated product has Not I sites on either end for insertion into the vector.

FIG. 5 is a photograph of RT-PCR products, run on a 1.0% agarose gel. The products derived from proteins transcribed from the FTL0552 region depicted in FIG. 1, to confirm loss of FTL0552 transcription in the F. tularensis LVS FTL0552 mutant and to determine the effect on downstream genes. Lanes 1 and 2 are amplicons using FTL0552 specific primers FTL0552-F and FTL0552-R, lanes 3 and 4 are replicons using mutS (house keeping gene) specific primers mutS-F and mutS-R, lanes and 6 show amplicons using lepB specific primers lepB-F and lepB-R, lanes 7 and 8 are amplicons using rnr specific primers rnr-F and rnrR. F. tularensis LVS RNA was used for the reactions in lanes 1, 3, 5, 7, and 13. FTL0552 mutant RNA was used for the reaction in lanes 2, 4, 8 and 14. Lanes 9-12 are water controls and lanes 13 and 14 are no reverse transcription controls.

FIG. 6 is a graph of FTL0552 mutant, showing growth defect under acellular conditions. The effect of FTL0552 deletion was assessed by growing the FTL0552 mutant and wild type F. tularensis LVS in Mueller Hinton broth. Aliquots were removed at the indicated times and the optical density measured at 550 nm (OD₅₅₀).

FIG. 7 is a graph showing a macrophage invasion assay of FTL0552 mutant and F. tularensis LVS on J774A.1 cells cells, using an MOI of 100 for 2 hours. After the infection, the cells were treated with 50 μg/ml gentamicin to kill the extracellular bacteria. At the indicated time points, cells were lysed, serially diluted, and plated on GC agar plates to determine colony forming units.

FIG. 8 is a graph showing a macrophage invasion assay of FTL0552 mutant and F. tularensis LVS using thioglycolate elicited peritoneal macrophages from BALD/c mice infected, at an MOI of 50 for 1.5 hours. The results are expressed as CFU/ml and represent means±standard errors of the means of the colon forming Unit counts (n=3 per time point). *P<0.001: **P<0.05 using the Student's T-Test.

FIG. 9 is a microscopic image of a macrophage invasion assay using fluorescently labeled F. tularensis GFP-LVS. The stained LVS illuminate as bright foci.

FIG. 10 is a microscopic image of a macrophage invasion assay using fluorescently labeled F. tularensis GFP-LVS. Grey arrows, outlined in white, indicate the stained bacteria are located inside the macrophages, whereas white arrows indicate stained bacteria are present on the surface of the cell. The macrophages were counterstained with PKH26 red fluorescence dye (Sigma, St Louis, Mo.). Insets: Confocal images confirming the localization of the bacteria within the cells.

FIG. 11 is a microscopic image of a macrophage invasion assay using fluorescently labeled F. tularensis FTL0552 mutant. The stained LVS illuminate as bright foci.

FIG. 12 is a microscopic image of a macrophage invasion assay using fluorescently labeled F. tularensis FTL0552 mutant. Grey arrows, outlined in white, indicate the stained bacteria are located inside the macrophages, whereas white arrows indicate stained bacteria are present on the surface of the cell. The macrophages were counterstained with PKH26 red fluorescence dye (Sigma, St Louis, Mo.). Insets: Confocal images confirming the localization of the bacteria within the cells.

FIG. 13 is a graph showing the FTLOS52 mutant is attenuated for virulence in mice and provides partial protection against lethal SchuS4 challenge. Survival of BALB/c (n=10) mice infected intranasally with 1×10⁴ CFU of FTL0552 mutant or F. tularensis LVS. Untreated mice were kept as controls. Results were expressed as Kaplan-Meier curves and P values determined using a Log-Rank test.

FIG. 14 is a graph showing the FTLOS52 mutant is attenuated for virulence in mice and provides partial protection against lethal SchuS4 challenge. Survival of BALB/c (n=10) mice infected intranasally with 1×10⁵ CFU of FTL0552 mutant or F. tularensis LVS. Untreated mice were kept as controls. Results were expressed as Kaplan-Meier curves and P values determined using a Log-Rank test.

FIG. 15 is a graph showing the FTL0552 mutant is attenuated for virulence in mice and provides partial protection against lethal SchuS4 challenge. Survival of C57BL/6 (n=10) mice infected intranasally with 1×10⁴ CFU of FTL0552 mutant or F. tularensis LVS. Untreated mice were kept as controls. Results were expressed as Kaplan-Meier curves and P values determined using a Log-Rank test.

FIG. 16 is a graph showing the FTL0552 mutant is attenuated for virulence in mice and provides partial protection against lethal SchuS4 challenge. Survival of C57BL/6 (n=10) mice infected intranasally with 1×10⁵ CFU of FTL0552 mutant or F. tularensis LVS. Untreated mice were kept as controls. Results were expressed as Kaplan-Meier curves and P values determined using a Log-Rank test.

FIG. 17 is a graph showing the FTL0552 mutant is attenuated for virulence in mice and provides partial protection against lethal SchuS4 challenge. Mice surviving 1×10⁵ CFU of FTL0552 mutant (n=10) were challenged with 1×10² CFU of SchuS4. Untreated mice were kept as controls. Results were expressed as Kaplan-Meier curves and P values determined using a Log-Rank test.

FIG. 18 is a graph showing the FTL0552 mutant is attenuated for virulence in mice and provides partial protection against lethal SchuS4 challenge. Mice surviving 1×10⁴ CFU of FTL0552 mutant (n=10) were boosted with 1×10⁵ CFU of FTL0552 mutant and challenged 30 days later with 1×10² CFU of F. tularensis SchuS4 strain. Untreated mice were kept as controls. Results were expressed as Kaplan-Meier curves and P values determined using a Log-Rank test.

FIG. 19 is a bar graph depicting FTL0552 mutant is rapidly cleared by BALB/c mice. BALD/c mice were inoculated intranasally with 5×10³ CFU of FTL0552 mutant or F. tularensis LVS. Four mice were killed at each indicated time point, and homogenates of the lungs were plated for the determination of bacterial burden. Results represent the means±standard errors of CFU counts (n=4 per time point). **P<0.01; ***P<0.001 (using the nonparametric Mann-Whitney test).

FIG. 20 is a bar graph depicting FTL0552 mutant is rapidly cleared by C57BL/6 mice. C57BL/6 mice were inoculated intranasally with 5×10³ CFU of FTL0552 mutant or F. tularensis LVS. Four mice were killed at each indicated time point, and homogenates of the lungs were plated for the determination of bacterial burden. Results represent the means±standard errors of CFU counts (n=4 per time point). **P<0.01; ***P<0.001 (using the nonparametric Mann-Whitney test).

FIG. 21 is a bar graph depicting FTL0552 mutant is rapidly cleared by BALB/c mice. BALD/c mice were inoculated intranasally with 5×10³ CFU of FTL0552 mutant or F. tularensis LVS. Four mice were killed at each indicated time point, and the numbers of bacteria were quantified in the liver of the mice at day 3 post-infection. Results represent the means±standard errors of CFU counts (n=4 per time point). **P<0.01; ***P<0.001 (using the nonparametric Mann-Whitney test).

FIG. 22 is a bar graph depicting FTL0552 mutant is rapidly cleared by C57BL/6 mice. C57BL/6 mice were inoculated intranasally with 5×10³ CFU of FTL0552 mutant or F. tularensis LVS. Four mice were killed at each indicated time point, and the numbers of bacteria were quantified in the liver of the mice at day 3 post-infection. Results represent the means±standard errors of CFU counts (n=4 per time point). **P<0.01; ***P<0.001 (using the nonparametric Mann-Whitney test).

FIG. 23 is a bar graph depicting FTL0552 mutant is rapidly cleared by BALB/c mice. BALD/c mice were inoculated intranasally with 5×10³ CFU of FTL0552 mutant or F. tularensis LVS. Four mice were killed at each indicated time point, and the numbers of bacteria were quantified in the spleen of the mice at day 3 post-infection. Results represent the means±standard errors of CFU counts (n=4 per time point). **P<0.01; ***P<0.001 (using the nonparametric Mann-Whitney test).

FIG. 24 is a bar graph depicting FTL0552 mutant is rapidly cleared by C57BL/6 mice. C57BL/6 mice were inoculated intranasally with 5×10³ CFU of FTL0552 mutant or F. tularensis LVS. Four mice were killed at each indicated time point, and the numbers of bacteria were quantified in the spleen of the mice at day 3 post-infection. Results represent the means±standard errors of CFU counts (n=4 per time point). **P<0.01; ***P<0.001 (using the nonparametric Mann-Whitney test).

FIG. 25 is a microscopic image showing the histological changes in H & E stained BALB/c mouse liver (n=4) 5 days after intranasal inoculation with 5×10³ CFU of F. tularensis LVS. Liver sections from sham-inoculated mice served as a control. Arrows indicate granulomas observed to be smaller in mice infected with FTL0552 mutant compared to LVS. Magnification 40×.

FIG. 26 is a microscopic image showing the histological changes in H & E stained BALB/c mouse liver (n=4) 5 days after intranasal inoculation with 5×10³ CFU of FTL0552 mutant. Liver sections from sham-inoculated mice served as a control. Arrows indicate granulomas observed to be smaller in mice infected with FTL0552 mutant compared to LVS. Magnification 40×.

FIG. 27 is a microscopic image showing the histological changes in H & E stained C57BL/6 mouse liver (n=4) 5 days after intranasal inoculation with 5×10³ CFU of F. tularensis LVS. Liver sections from sham-inoculated mice served as a control. Arrows indicate granulomas observed to be smaller in mice infected with FTL0552 mutant compared to LVS. Magnification 40×.

FIG. 28 is a microscopic image showing the histological changes in H & E stained C57BL/6 mouse liver (n=4) 5 days after intranasal inoculation with 5×10³ CFU of FTL0552 mutant. Liver sections from sham-inoculated mice served as a control. Arrows indicate granulomas observed to be smaller in mice infected with FTL0552 mutant compared to LVS. Magnification 40×.

FIG. 29 graphs the levels of proinflammatory cytokines (TNF-α) resulting from insult of FTL0552 mutant and F. tularensis LVS. Following intranasal inoculation of BALB/c mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 30 graphs the levels of proinflammatory cytokines (MCP-1) resulting from insult of FTL0552 mutant and F. tularensis LVS. Following intranasal inoculation of BALB/c mice with 5×10³ CFU of FTL0552 mutant or F. tularensis LVS, lung homogenates were prepared. and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 31 graphs the levels of proinflammatory cytokines (IFN-γ) resulting from insult of FTL0552 mutant and F. tularensis LVS. Following intranasal inoculation of BALB/c mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 32 graphs the levels of proinflammatory cytokines (IL-6) resulting from insult of FTL0552 mutant and F. tularensis LVS. Following intranasal inoculation of BALB/c mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 33 graphs the levels of proinflammatory cytokines (IL-12) resulting from insult of FTL0552 mutant and F. tularensis LVS. Following intranasal inoculation of BALB/c mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 34 graphs the levels of proinflammatory cytokines (TNF-α) resulting from insult of FTL0552 mutant and F. tularensis LVS. Following intranasal inoculation of C57BL/6 mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 35 graphs the levels of proinflammatory cytokines (MCP-1) resulting from insult of FTL0552 mutant and F. tularensis LVS. Following intranasal inoculation of C57BL/6 mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 36 graphs the levels of proinflammatory cytokines (IFN-γ) resulting from insult of FTL0552 mutant and F. tularensis LVS. Following intranasal inoculation of C57BL/6 mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 37 graphs the levels of proinflammatory cytokines (IL-6) resulting from insult of FTL0552 mutant and F. tularensis LVS. Following intranasal inoculation of C57BL/6 mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

FIG. 38 graphs the levels of proinflammatory cytokines (IL-12) resulting from insult of FTL0552 mutant and F. tularensis LVS. Following intranasal inoculation of C57BL/6 mice with 5×10³ CFU of FTL0552 mutant or F. tularensis LVS, lung homogenates were prepared, and levels of proinflammatory cytokine levels were measured using Cytometric Bead Array (BD Pharmingen, San Diego, Calif.). Results represent the means±standard errors of the means of the cytokine concentrations (n=4 mice per time point). *P<0.05; **P<0.01; ***P<0.001 (by one-way ANOVA followed by Bonferroni's correction).

### DETAILED DESCRIPTION OF THE PREFERRED EMBODIMENT

Bacterial PhoP protein sequences were retrieved from GenBank (NCBI) and aligned using the PileUp Program for aligning multiple sequences from the Genetics Computer Group (GCG Wisconsin Package Version 10). The consensus sequence, generated from the alignment of bacterial PhoP proteins, was used to search the TIGR Comprehensive Microbial Resource (CMR) with the PhoP consensus sequence. A gapped BLAST search of the SchuS4 and LVS genomes with the consensus sequence identified candidate phoP genes, with 26% identical deduced amino acid sequences. The homologues contain both a response-regulator receiver domain and a transcriptional regulatory protein domain, consistent with response-regulator proteins. Nearly identical coding sequences were identified in both the SchuS4 (FTL1557c locus) and in LVS (FTL0552 locus) genomic databases.

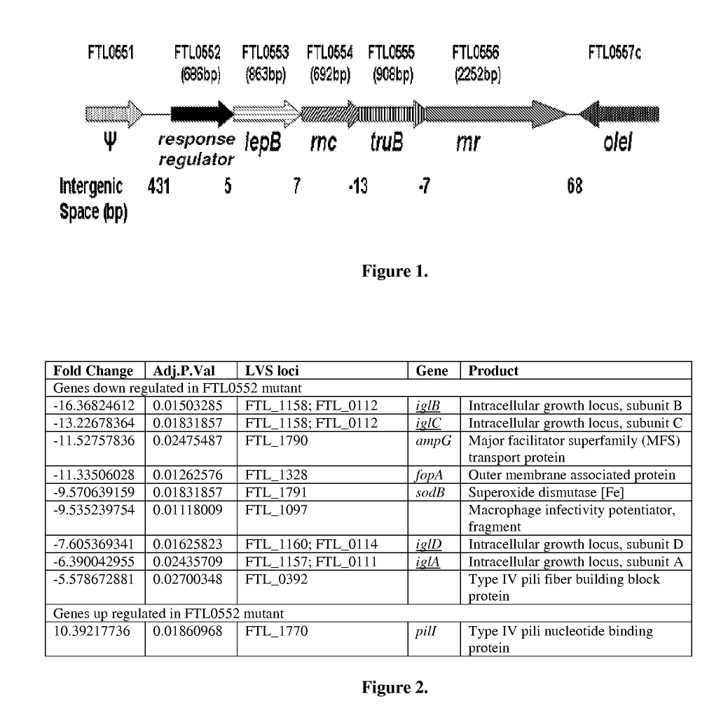

The FTL0552 locus has motifs consistent with response-regulator proteins, containing both a response-regulator receiver domain and a transcriptional regulatory protein domain. The locus is annotated as a two-component response-regulator, but no sensor kinase gene is immediately upstream or downstream of this gene, seen in FIG. 1. The gene arrangement of adjacent genes and the deduced amino acid sequence homology of this putative response-regulator gene are highly conserved between LVS and the virulent F. tularensis SchuS4 strain, where FTL1557c shows only one conservative amino acid change in residues 21-228. FTL0552 is the first gene in a series of five that appear to form a single operon with minimal intergenic spaces or in some cases overlapping genes, seen in FIG. 1. RT-PCR revealed that FTL0552 and the four downstream genes are transcribed as one complete transcript, indicating that FTL0552 is the first gene of a five-gene operon, transcribed from the same promoter (data not shown).

RNA was extracted from the LVS and FTL0552 mutant (described below) using protocol from the RNAprotect Bacteria Reagent Handbook (Qiagen, Valencia, Calif.), followed by purification using the RNeasy Mini Kit Purification of Total RNA from Bacterial Lysate protocol. cDNA was synthesized from 1 μg of total RNA using 5 μg of random hexamers (Amersham Biosciences, Piscataway, N.J.) and Superscript III (Invitrogen) in a standard reverse transcription reaction. Amino allyl dUTPs were incorporated (2.5 μM of each dATP, dCTP, dGTP, and dUTP) and cDNA purified using Zymo DNA purification columns (Zymo Research Corp., Orange, Calif.). Samples were labeled with Cy5 (red) fluorophores and references labeled with Cy3 (green) fluorophores (Amersham Biosciences). Unincorporated fluorophore was quenched with 5 μl of 4M hydroxylamine, followed by 15 min incubation in the dark. Unincorporated dye was removed with Zymo DNA purification columns and cDNA eluted with 19 μl Tris-EDTA, 2 μl of 20 mg/ml yeast tRNA (Invitrogen), 4.25 μl of 20×SSC, and 0.75 μl of 10% sodium dodecyl sulfate (SDS). Probes were denatured for 2 min at 99° C., spun at 17,900×g, cooled at room temperature and added to the arrays. The samples and arrays were incubated at 60° C. for 14 hr. The arrays were washed in four increasing stringency washes (i) 2×SSC-0.03% SDS, (ii) 2×SSC, (iii) 1×SSC, and (iv) 0.2×SSC. The microarrays were scanned and analyzed using a Gene Pix 4000A scanner and GENEPIX5.1 software (Axon Instruments, Redwood City, Calif.). Normalized data were collected using the Stanford Microarray Database. Spots with at least 70% good data across the experiment were included for analysis. The ratios of the red channels to green channels for each spot were expressed as log₂ (red/green) and used for hierarchical clustering using the CLUSTER program. Results were visualized using the TREEVIEW program. Using data from all of the microarrays were analyzed using the Significance Analysis for Microarrays (SAM) program. v. 1.21. A calculated false discovery rate of <1% was used to assign significance, and a two-fold cutoff in the change of expression level imposed.

Gene microarray analysis revealed 148 genes regulated by FTL0552, seen in FIG. 2, and identified by a 4-fold or greater difference between F. tularensis LVS parent strain and FTL0552 mutant. The majority (75%) of the genes are down regulated in the FTL0552 mutant, indicating they are FTL0552 activated genes. Among the down regulated genes are the intracellular growth loci (iglA, iglB, iglC and iglD), located in the FPI and shown to be essential for intracellular survival. Other down regulated genes include a macrophage infectivity potentiator fragment, a type IV pili fiber building block protein, ampC, fopA, and, superoxide dismusase gene sodB. The type IV pili fiber building block protein is involved in bacterial attachment and invasion of host cells. Citrobacter freundii AmpC is a cytoplasmic membrane protein gene required for induction of β-lactamase and recently-identified permease required for recycling murein tripeptide and uptake of anhydro-muropeptides. FopA is an outer membrane protein specific to F. tularensis. SodB encodes a FeSOD protein in F. tularensis LVS, essential for oxidative stress bacterial survival. Up regulated genes in the FTL0552 mutant include hypothetical proteins and pseudogenes, two transposases and, a type IV pili nucleotide binding protein, pilT.

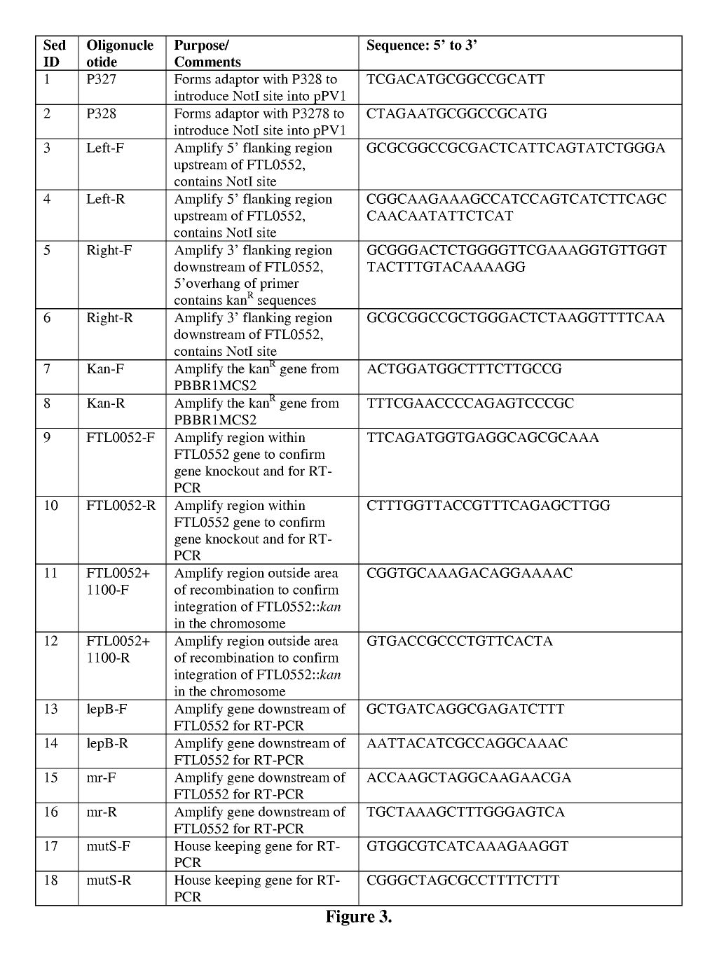

A plasmid derivative of pPV was constructed with an added Not I restriction site and erythromycin resistance (Erm²) substituted for chloramphenicol resistance (Cm^{R}). pPV shuttle vector plasmid (Cm^{R}AP^{R}) transformed E. coli DH5a recipient to chloramphenicol resistance. pPV DNA was prepared from an E. coli DH5a transformant, and a Not I cloning site introduced. Oligonucleotides P327 and P328, listed in FIG. 3, were annealed to produce adaptors. pPV was cleaved with Sal I and Xba I and the P327/P328 construct ligated into the pPV vector. The presence of the Not I site was confirmed, creating the pPVNot plasmid with unique Not I, Sal I, and Xba I sites. A derivative of pPVNot was constructed with the Cm^{R} marker replaced with Erm^{R}. pPVNot was partially digested with HindIII, religated and transformed into E. coli DH5a with selection for ampicillin resistance. Colonies were screened for the loss of chloramphenicol resistance and the loss of the 3.2 kb Hind III fragment that carries Cm^{R}. This plasmid (Ap^{R},Cm^{S}) was linearized by partial digestion with Hind III and ligated to the ermC gene from pKS65. Transformants were scored for ampicillin and erythromycin resistance on LB agar plates containing 200 μg/ml erythromycin and 100 μg/ml ampicillin. The resulting plasmid was designated pPVNot/Erm and this plasmid was used for gene transfer into F. tularensis LVS.

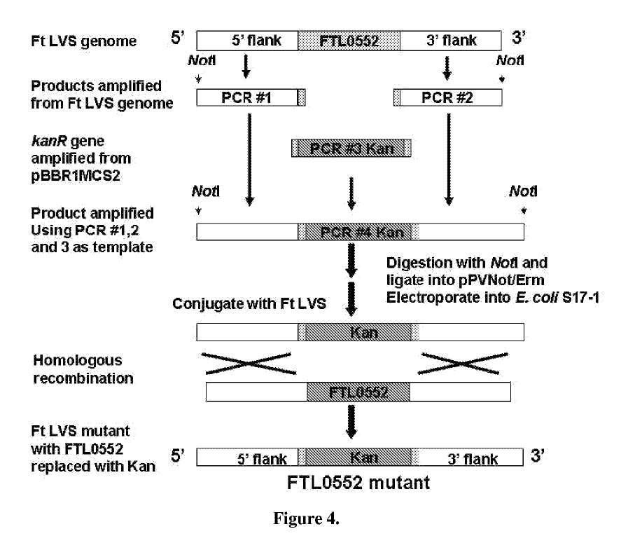

A knockout plasmid was generated using PCR products. The flanking sequence of approximately 700 bp upstream and 700 bp downstream of FTL0552 were amplified from F. tularensis LVS genomic DNA in two separate PCR reactions, using EasyStart™ 100 reagents. Primer sequences for the 5′ flanking regions, Left-F and Left-R, and the 3′ flanking sequences, Right-F and Right-R, are shown in FIG. 3. A third PCR product consisting of the kanamycin resistance gene was amplified from pBBRIMCS2 using Kan-F and Kan-R primers. All three products were gel-purified using Geneclean Turbo and used as templates to generate a single product with upstream and downstream flanking regions of FTL0552 and coding regions from nucleotides 26-645 were replaced with kan^{R}. This PCR product was generated using primers Left-F and Right-R and is flanked by Not I restriction sites creating a 2349 base pairs-long PCR product. The PCR product was cleaved with Not I and ligated into the Not I site of pPVNot/Ertu and transformed into E. coli S 17-1. The plasmid construct was confirmed by digestion with Not I and then transformed into E. coli S 17-1. See FIG. 4.

A FTL0552 plasmid coding sequence interrupted by the kan^{R} gene was created through allelic replacement (Golovliov, Sjosteck et al. 2003) and introduced into E. coli S 17-1 for mobilization and transfer to LVS. FTL0552 mutant was transferred from E. coli S 17-1 to F. tularensis LVS via bacterial conjugation, depicted in FIG. 4, followed by selection on GC agar plates containing 100 μg/mL polymyxin B and 25 μg/mL kanamycin. Isolated colonies were spotted on plates containing 5% sucrose, kanamycin (25 μg/ml), and polymyxin B 100 μg/ml) for counter selection with sacB. Colonies that were both kanamycin and sucrose resistant were verified by PCR and used for further analysis.

RNA integrity was confirmed using RNA isolated from Mueller Hinton broth cultures of F. tularensis LVS parental strain and FTL0552 mutant grown to log phase (OD₅₅₀ of 0.600). The RNA was isolated using TRIzol reagent (Invitrogen. Carlsbad, Calif.), RNeasy clean-up protocol (Qiagen, Valencia, Calif.) and a 15 minute DNase digestion (Qiagen, Valencia, Calif.). RNA concentration was assessed at OD₂₆₀ and OD₂₈₀, and the integrity of the 23S and 16S rRNA verified on a 0.7% agarose gel.

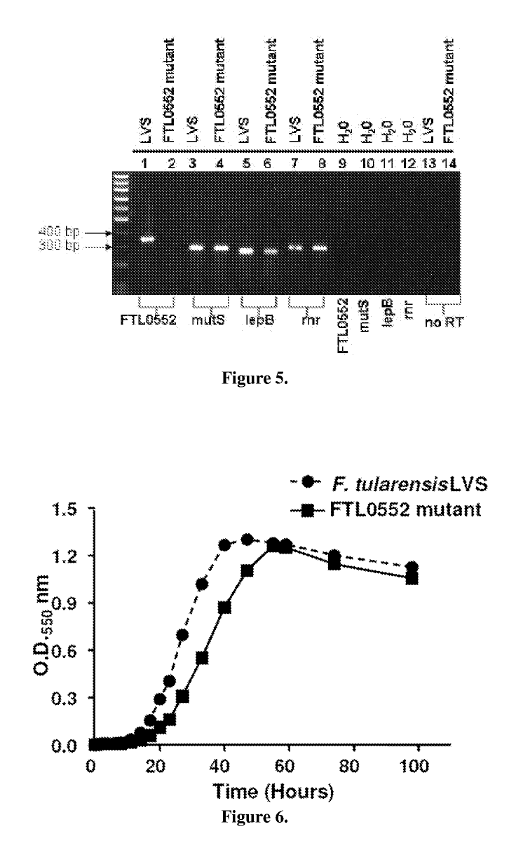

cDNA was synthesized from the RNA isolated from both the F. tularensis LVS Parental strain and FTL0552 mutant. Primers were designed for the second and fifth gene immediately downstream of FTL0552. See FIG. 1. RT-PCR was performed using the One-Step RT-PCR kit (Qiagen, Valencia, Calif.), at 50° C. for 30 minutes and 95° C. for 15 minutes; followed by 30 cycles of 94° C. for one minute, 55° C. for 1 minute and 68° C. for 1 minute; then a final elongation step of 72° C. for 10 minutes. RT-PCR primers FTL0552-F and FTL0552-R (specific for FTL0552), lepB-F and lepB-R (lepB), primers mr-F and rnr-R (mr), and mutS-F and mutS-R (reference house keeping gene mutS) were used. See FIG. 3. The resultant products were run on a 1.0% agarose gel, showing 352 bp for FTL0552, 288 bp for lepE, 314 bp for rnr, and 304 bp for mutS, seen in FIG. 5.

LepB and rnr were selected to determine the effects of knocking out FTL0552 on transcription of downstream genes within the potential operon. The results confirmed that FTL0552 was not transcribed in the mutant strain, while the two downstream genes examined were transcribed at levels similar to those seen in LVS, seen in FIG. 5. Therefore, the phenotype seen in the FTL0552 knockout mutant is not a result of polar effects but is directly attributed to the mutation in FTL0552 and ultimately to genes outside the putative operon regulated by FTL0552.

F. tularensis LVS was grown on agar consisting of GC agar base (Remel, Lenexa, Kans.) supplemented with 5% fetal bovine serum, 1% bovine hemoglobin and 1% IsoVitaleX™ (Becton Dickinson, Sparks, Md.) incubated at 37° C. The F. tularensis LVS FTL0552 mutant was grown on the same agar with the addition of 50 μg/ml kanamycin. For some experiments, bacteria were grown in Mueller Hinton II (MH) broth (Becton Dickinson, Sparks, Md.) supplemented with 0.1% glucose, 2% IsoVitaleX™ and 33 μM ferric pyrophosphate. For bacterial growth analysis. MH broth cultures were incubated at 37° C. with aeration and the OD₅₀ was measured at various time points.

### Example 1

Immortalized mouse macrophage cell lines (J774A.1) were cultured in Dulbecco's Modification of Eagles Medium (DMEM), supplemented with 10% fetal bovine serum (FBS), 4.5 g/mL glucose and L-glutamine, and 50 μg/ml penicillin-streptomycin. Mouse peritoneal exudate cells (PEC) were collected from thioglycolate treated BALB/c mice, washed and resuspended in medium containing 10% FBS, 0.33 μl/ml 2-mercaptoethanol, and L-glutamine.

J774A.1 mouse macrophage cells were seeded into a 24 well tissue culture plate at 6×10⁴ cells/well and incubated overnight at 3° C. with 5% CO₂. F. tularensis LVS parental and FTL0552 mutant strain were suspended in DMEM, added to each well at an MOI of 100, and allowed to infect for 2 hr at 37° C. in 5% CO₂. Following infection, the cells were washed twice with PBS to remove extracellular bacteria. Gentamicin (50 μg/ml) was added to the wells and then incubated for 1 hr. Thereafter, the cells were washed once with PBS and media containing gentamicin (2 μg/ml) was added to the wells. At 0, 24, 48, 72 hrs the cells were washed with PBS and lysed with 0.1% sodium deoxycholate. Viable counts were performed by plating serial 10-fold dilutions of the lysates on supplemented GC agar plates and incubating at 37° C., performed in triplicate. Infection of peritoneal exudate cells (PECs) was performed with Peritoneal cells (PC), collected from thioglycolate-treated BALB/c mice, seeded into a 96 well tissue culture plate at a density of 1×10⁵ cells/well and incubated overnight at 3° C. in 5% CO₂. F. tularensis LVS and FTL0552 mutant bacteria were added to each well at an MOI of 50, allowed to infect for 1.5 hrs, then washed twice with HBSS and incubated for 1 hr in medium containing gentamicin (50 μg/ml). Thereafter, the cells were washed once with HBSS and cultured in medium containing gentamicin (2 μg/ml). At 0, 12, 24, and 48 hrs the cells were washed with HBSS and lysed with 0.1% saponin. Viability counts were performed by plating serial dilutions as before.

FTL0552 mutant was stained with PKH67 green fluorescent cell linker mini kit (Sigma, St. Louis, Mo.), as per the manufacturer's instructions. LVS transformed with a GFP plasmid was used a control. 1×10⁴ MH-S (murine alveolar macrophage cell line) were seeded on a sterile Lab-Tek chamber slide (Nalge Nunc International, Rochester, N.Y.) and incubated for 12 hr at 37° C. in cells in RPMI-1640 supplemented with 10% FBS. The cells were infected with the labeled FTL0552 or the GFP-LVS at an MOI of 100 and incubated at 37° C. for 15, 30, and 45 minutes. The cells were washed twice with sterile PBS and fixed with 3% paraformaledehyde. MH-S cells were counterstained with PKH26 red fluorescence cell linker mini kit (Sigma St. Louis. MO) as per the manufacturer's instructions, and washed and mounted. For confocal microscopy, 0.5 μM Z sectioning was performed on a under a C-Apochromat confocal microscope objective at 40× magnification with 1.2 W corrections and visualized in channel-1 at 500-550 IR and channel-2 at 565-615 IR. The images were analyzed using LSM5 image browser software version 3.2.0.115 (Carl Ziess, Biocompare Inc., San Francisco, Calif.).

### Example 2

Six to eight week-old BALB/c and C57BL/6 mice (Taconic, Germantown, N.Y.) were housed in the Animal Resource Facility at Albany Medical College and used for screening of the FTL0552 mutant. Prior to intranasal (i.n.) inoculation with F. tularensis LVS or F. tularensis LVS FTL0552 mutant, the mice were deeply anesthetized via intraperitoneal injection of a cocktail of Ketamine (20 mg/ml) and Xylazine (1 mg/ml). The mice (n=10 for each group) were infected i.n. with 1×10⁴ or 1×10⁵ CFU of LVS or FTL0552 mutant in 20 μl PBS (10 μl per nare). The mice were monitored closely for morbidity and mortality for a period of 21-30 days post-infection and the median survival time (MST) was calculated for each group of mice. Mice that survived the initial infection dose of 1×10⁵ CFU of FTL0552 mutant were challenged with 1×10² CFU of the virulent F. tularensis SchuS4 strain. All mice that survived 1×10⁴ CFU of FTL0552 mutant were boosted with 1×10⁵ CFU of FTL0552 mutant and challenged 30 days later with 1×10² CFU of SchuS4. Actual numbers of bacteria were determined by plating the inoculum after primary infection and challenge. All the SchuS4 challenge experiments were carried out in the ABSL-3 facility of the Albany Medical College following standard operating procedures and conformed to the animal procedures approved by Institutional Animal Care and Use Committee guidelines.

A growth comparison between the LVS parental strain and the FTL0552 mutant was performed to assess the effects of knocking out FTL0552 on the bacteria's ability to grow in an acellular environment. Isolated colonies of F. tularensis LVS were visible on supplemented GC agar plates in 24 hours, whereas isolated colonies from the FTL0552 mutant required 38-42 hours. Overnight broth cultures were incubated at 37° C. with aeration for a period of 4 days and OD₅₀ readings were measured at several time points during the course of growth. Growth of the FTL0552 mutant reached the same cell density as the parental strain of LVS, however, at a slightly slower rate. See FIG. 6. The mutant required an additional 6-8 hours to reach stationary phase of growth, compared to parental strain LVS.

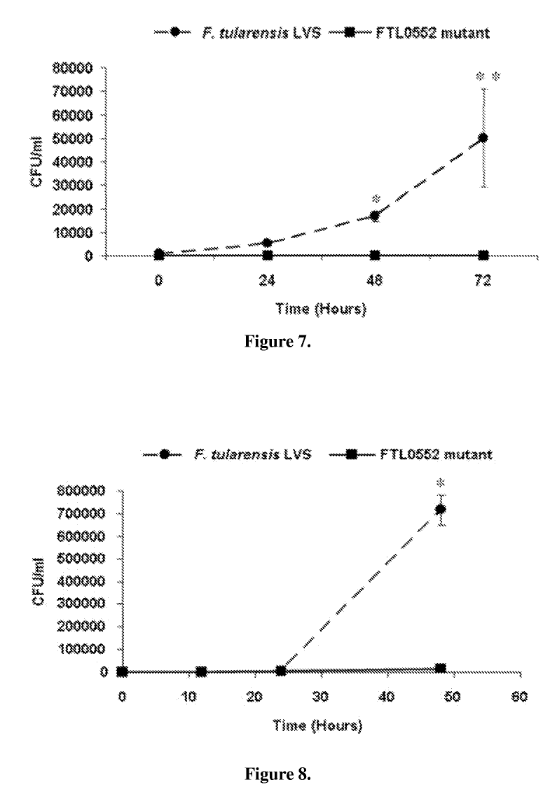

The ability of the mutant to invade and replicate within mouse macrophages was assessed using J774A.1 cells and mouse peritoneal macrophages. The cells were infected with the parental strain LVS or FTL0552 mutant LVS at an MOI of 100 for J774A.1 cells and an MOI of 50 for peritoneal cells for 2 hrs and 1.5 hrs, respectively. At the indicated time points, viable counts were performed by lysing the cells and incubating serial dilutions on GC agar plates. Over a period of 72 hrs, F. tularensis LVS was able to invade and replicate within 3774A.1 cells, increasing approximately 50-fold. However, F. tularensis LVS FTL0552 mutants provided significantly lower numbers of viable bacteria, shown in FIG. 7. Viable bacteria from PCs infected with the FTL0552 mutant were recovered, showing <20-fold increase, which showed significantly less replication than the parental strain at over >700-fold increase, seen in FIG. 8.

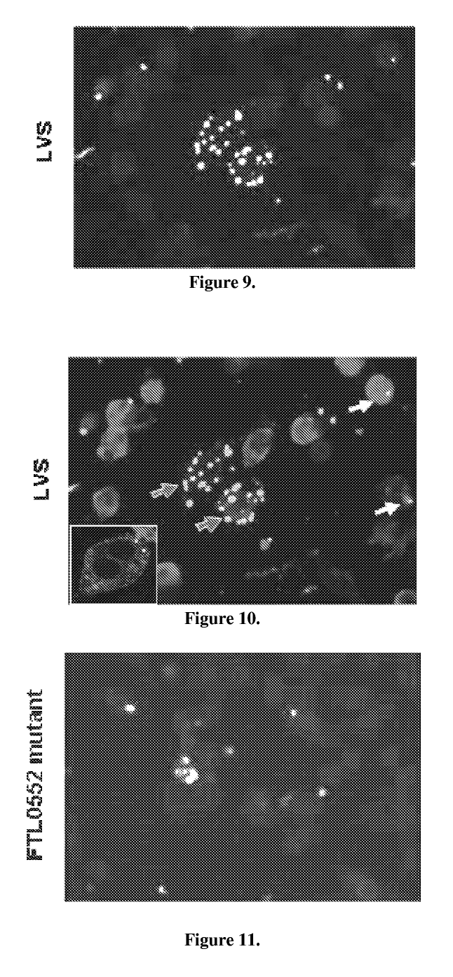

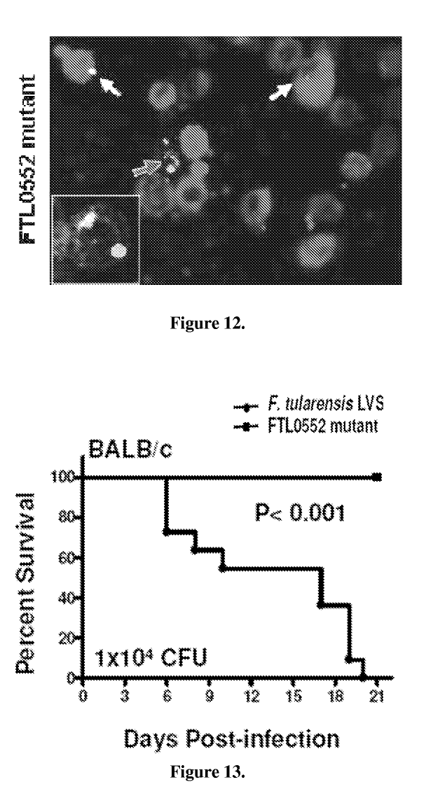

The inability of FTL0552 to invade the macrophages was further investigated. After 30 min of incubation with the labeled bacteria, the majority of MH-S cells infected with GFP-LVS harbored 2-10 bacteria per cell, where cells infected with FTL0552 mutant contained only 1-3 bacteria per cell, seen in FIGS. 9 to 12. Confocal microscopy confirmed that the bacteria indicated with a grey arrow outlined in white were localized inside the cells whereas, bacteria that took the counter stain, indicated with a full white arrow, were localized on the surface of the macrophages (FIGS. 10 and 12, Insets).

### Example 3

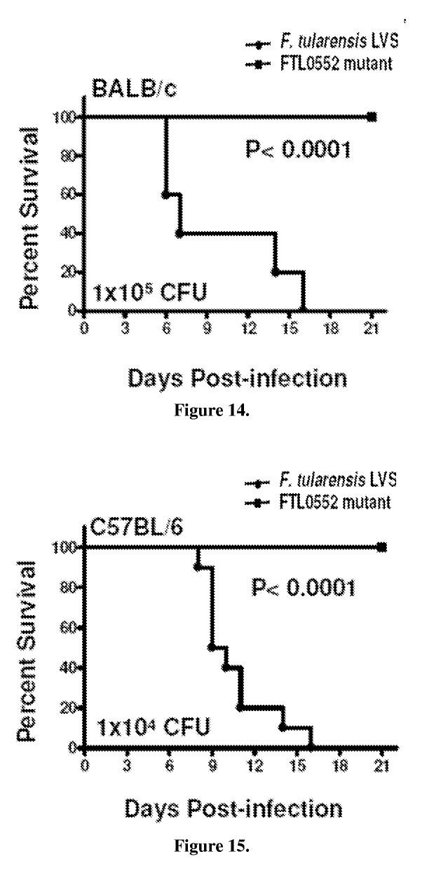

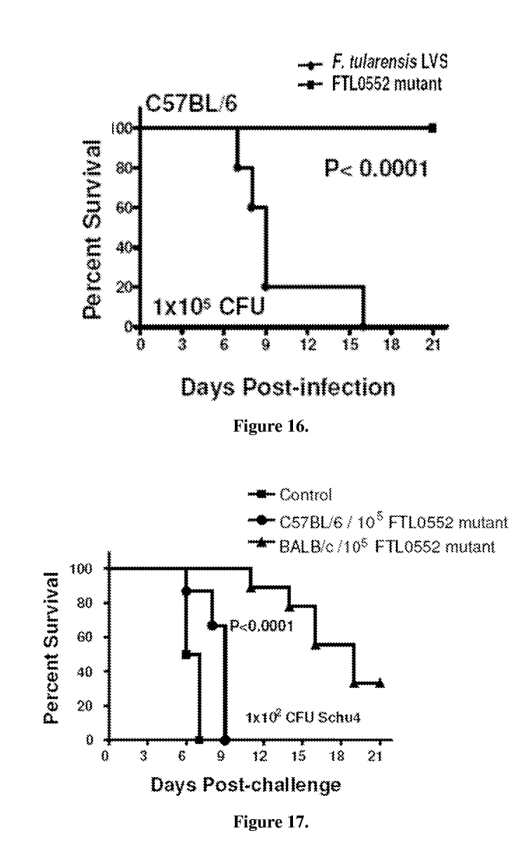

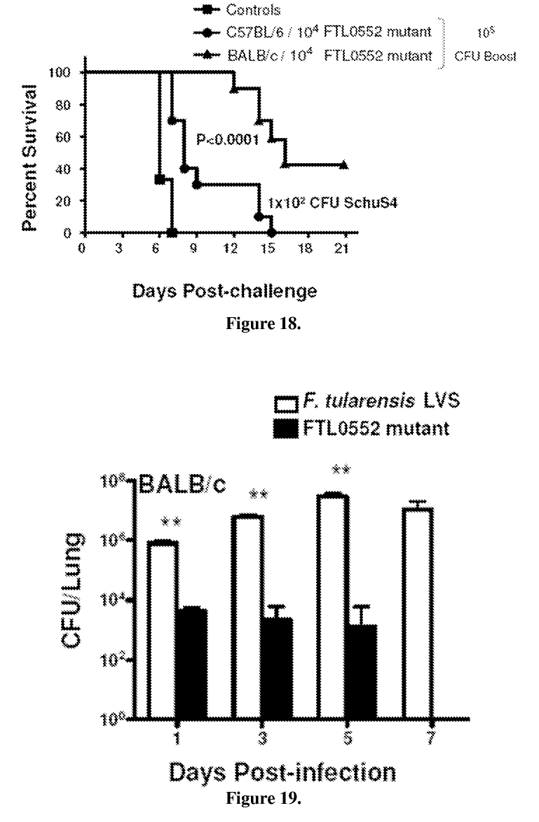

FTL0552 mutant was inserted into BALB/c and C57BL/6 mice to assess the effect of the deletion on virulence. Both strains of mice (n=10) were infected with 1×10⁴ or 1×10⁵ CFU of F. tularensis LVS and succumbed by days 17-19 post infection. Conversely, mice infected with similar doses of FTL0552 mutant survived to at least day 21 post-infection, depicted in FIGS. 13 to 16. Survival mice for the primary infective dose were challenged with the SchuS4 strain. C57BL/6 mice had an extended MST of 9 days, compared to 6.5 days for the naive challenged mice. In the BALB/c mice challenged with SchuS4, the first death was recorded at day 12 post-challenge and 30% (3/10) of the mice survived the challenge dose out to at least 45 days post-challenge when the experiment was terminated. See FIG. 17. The C57BL/6 mice appear resistant to the protection afforded the BALB/c mice. The other groups of mice that survived initial infection of FTL0552 were subsequently boosted and challenged with 1×10² CFU of SchuS4. Although the MST for C57BL/6 these mice was not altered following challenge, 40% of the mice survived until day 21 post-challenge and the others had a significant delay in time to death, seen in FIG. 18. These results show that the FTL0552 mutant is not only highly attenuated for virulence in mice, but also retains its antigenic potential and provides partial protection against virulent SchuS4 challenge.

A time course experiment was conducted to determine the kinetics of bacterial clearance. BALB/c and C57BL/6 mice were infected i.n. with 5×10³ CFU of either LVS or FTL0552 mutant. A group of four mice each was sacrificed at days 1, 3, 5, and 7 post-infection. Lung, liver, and spleen were collected aseptically and bacterial burdens were quantified. The lungs from infected mice were inflated with sterile PBS and collected aseptically in PBS containing a protease inhibitor cocktail (Roche Diagnostics, Indianapolis, Ind.). Livers and spleens were also collected in a similar fashion. The organs were subjected to mechanical homogenization using a Mini-BeadBeater-8™ (BioSpec Products Inc., Bartlesville, Okla.). Tissue homogenates were spun 1000×g for 10 sec and supernatants diluted 10-fold in sterile PBS. 10 μl of each dilution were spotted onto chocolate agar plates in duplicate and incubated at 37° C. for 2-3 days. The number of colonies on the plates were counted and expressed as CFU/gram of tissue.

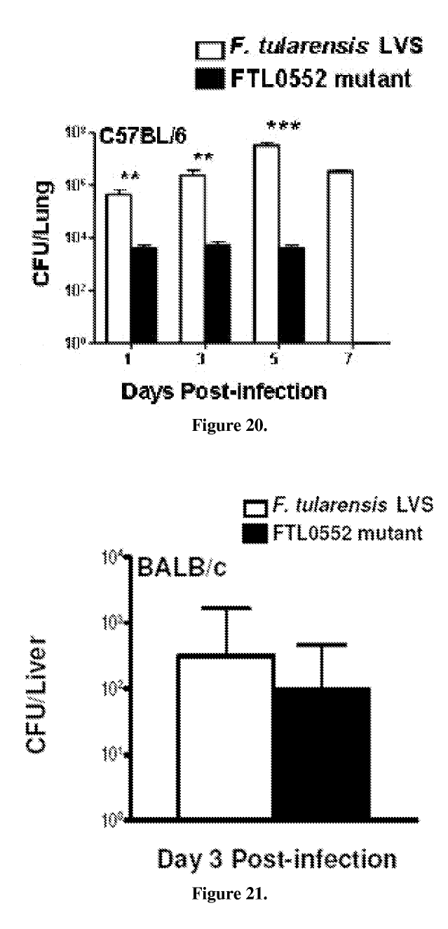

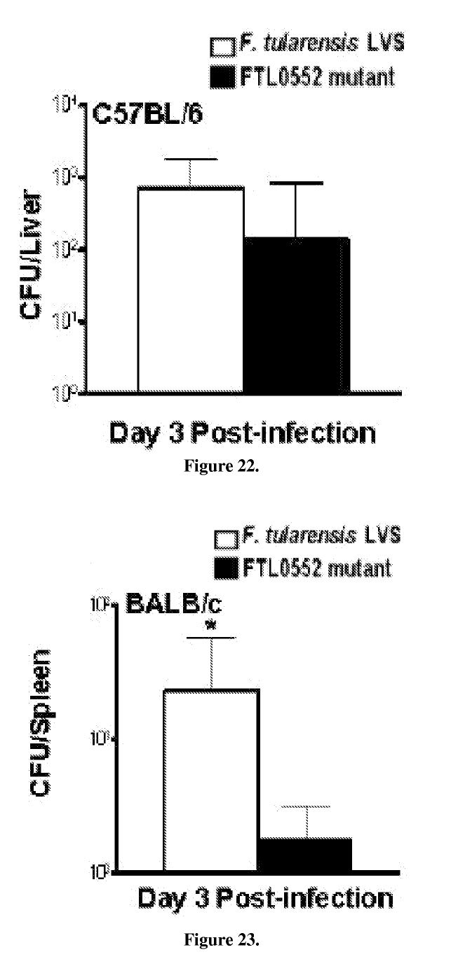

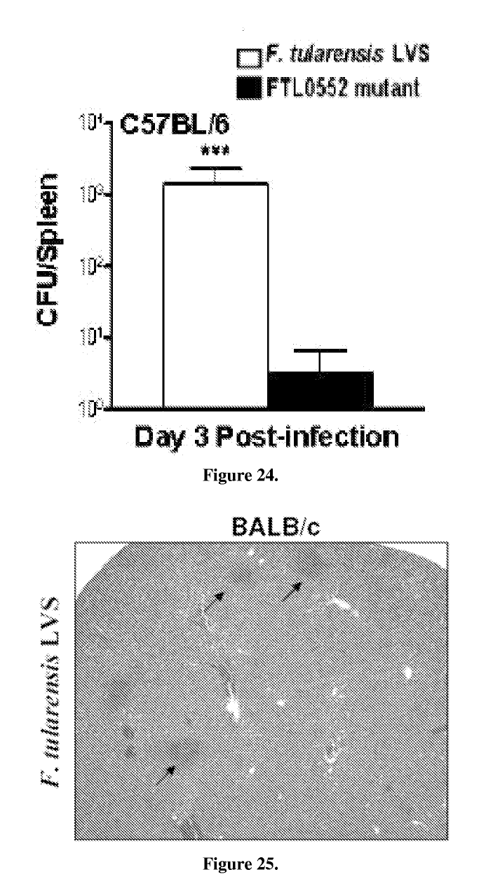

The clearance kinetics of the FTL0552 mutant in mice was determined. An identical pattern of bacterial kinetics was observed in both BALB/c and C57BL/6 mice. At days 1, 3, and 5 post-infection, bacterial numbers were significantly lower in the lungs of FTL0552 mutant-infected mice compared to LVS infected mice. By day 7 post-infection, bacteria were completely eliminated front the lungs of FTL0552 mutant-infected mice, seen in FIGS. 19 and 20. No significant differences were observed in bacterial numbers in LVS or FTL0552 mutant infected mice in the liver, at day 3 post-infection, seen in FIGS. 21 and 22, but at subsequent time points no bacteria were detected in FTL0552 mutant infected mice. Significantly lower numbers of FTL0552 mutant bacteria disseminated to the spleen, as compared to the liver, and no detectable bacteria were seen day 5 and 7 post-infection. See FIGS. 23 and 24. These results corroborate attenuated virulence of FTL0552 mutant observed in mice and suggest that attenuation of FTL0552 mutant may be due to rapid clearance and significantly diminished dissemination.

The lungs, livers, and spleens from infected mice were excised at days 1, 3, 5, and 7 post-infection and fixed with 10% neutral buffered formalin. Tissues were processed using standard histological procedures and 5 μm paraffin sections were stained with hematoxylin-eosin and examined by light microscopy. Lesions in the lungs, livers, and spleens of FTL0552 mutant and LVS infected mice appeared as early as 3 days post-infection and subsequently became more extensive by days 5 and 7 post-infection. See FIGS. 25 to 28, other data not shown.

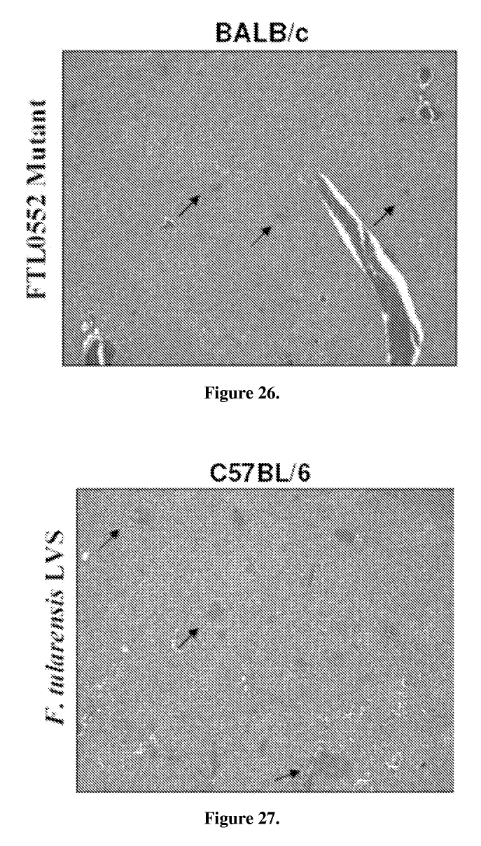

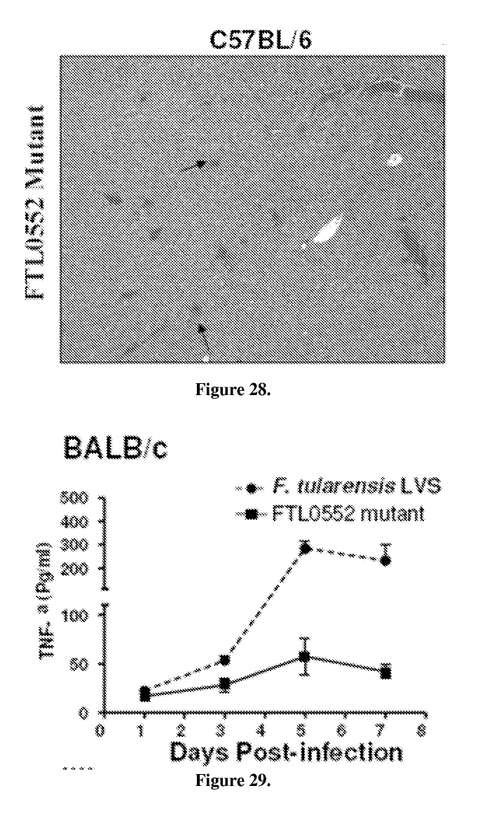

Lesions in the lungs of LVS infected mice consisted mostly of multifocal bronchopneumonia and show lymphocytic to neutrophilic peribronchial and perivascular inflammation. However, these lesions were less severe and localized to very discrete areas in the lungs of FTL0552 mutant infected mice. Livers from LVS infected mice showed numerous multifocal neutrophilic to lymphocytic granulomas that became larger as the infection progressed, seen in FIGS. 25 and 27. In FTL0552 mutant infected mice, few and very small granulomas were observed, as depicted in FIGS. 26 and 28. Lesions in the spleen consisted of multifocal to coalescing areas of neutrophilic infiltration in the red pulp, enlargement of the marginal zones, and extensive proliferative responses in the germinal centers. However, the splenic tissue in FTL0552 mutant-infected mice appeared to be normal and no inflammatory changes were observed. The results indicate that although the FTL0552 mutant is rapidly cleared from tissue, it still induces a degree of inflammation and mild pathological lesions in the lungs and liver.

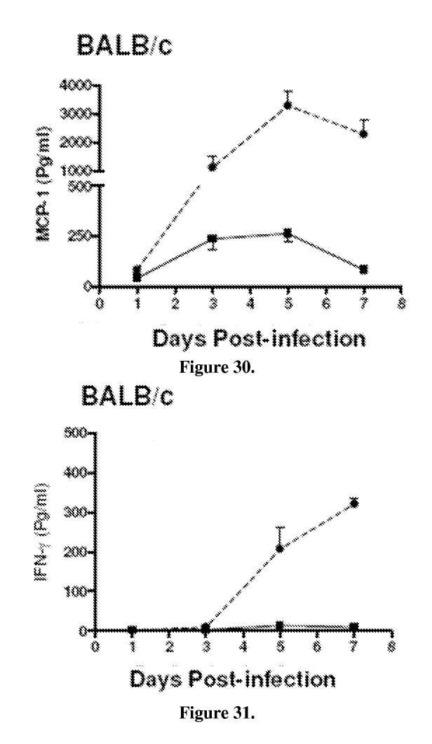

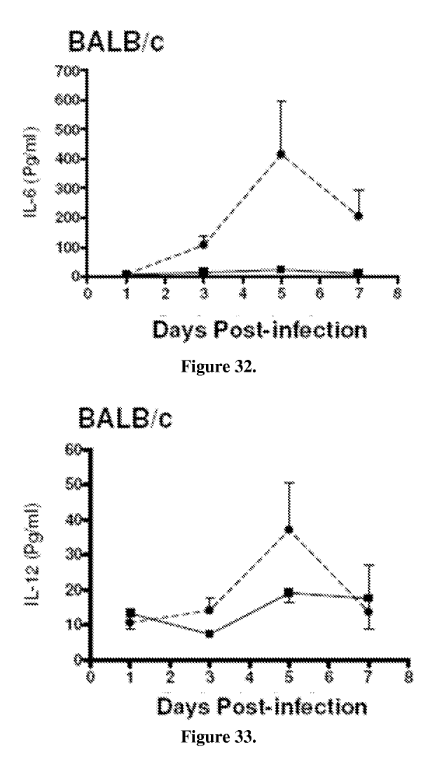

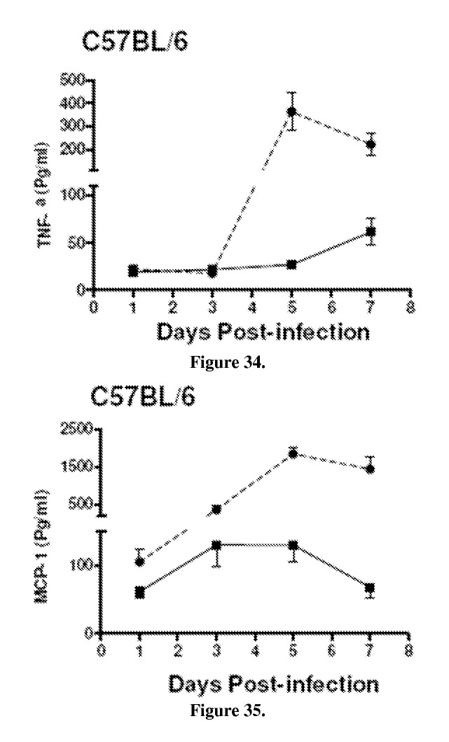

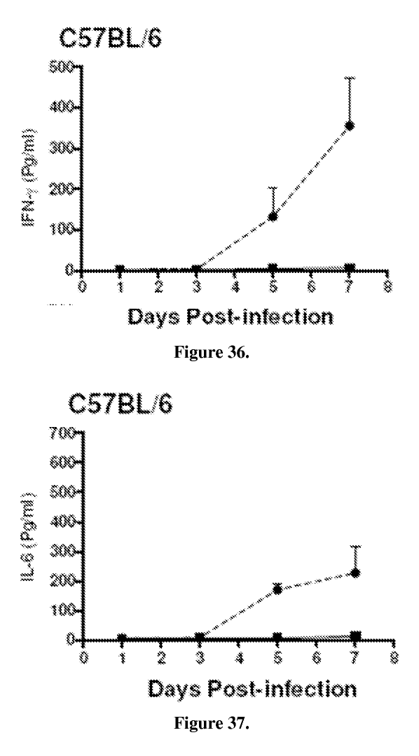

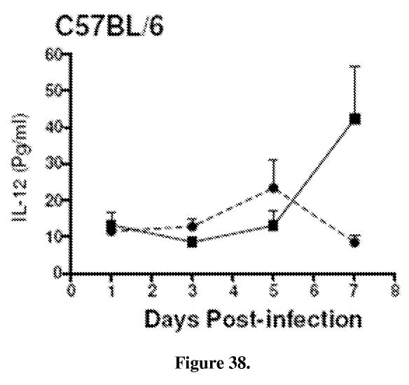

Cytokine production, in response to F. tularensis infection, is responsible for severe histopathological lesions observed in the lungs, livers and spleens of infected mice. The levels of inflammatory cytokines, such as interferon-gamma (IFN-γ), tumor necrosis factor-alpha (TNF-α). interleukin-6 (IL-6), monocyte chemoattractant protein (MCP-1) and interleukin 12 (IL-12) in the lung homogenates of C57BL/6 and BALB/c mice infected either with LVS or FTL0552 mutant. LVS infected mice had significantly elevated levels of all cytokines except IL-12 at days 5 and 7 post-infection, shown in FIGS. 29 through 38. The levels of IL-6 and IFN-γ in FTL0552 mutant-infected mice were below detection levels whereas elevated levels of IL-12 were seen at day 7 post-infection. The results suggest that lowered cytokine production results in less severe pathology in the lungs of FTL0552 mutant infected mice.

F. tularensis LVS mutant, with deleted FTL0552, was shown to be defective for survival in both mouse 3774A.1 cells and peritoneal macrophages. This mutant was completely attenuated in both BALB/c and C57BL16 mice at doses up to 1×10⁵ CFU, and was able to provide some protection against challenge with the virulent SchuS4 strain. Mice infected with the FTL0552 mutant exhibited reduced levels of pro-inflammatory cytokine production, and reduced histopathology in affected tissues, and reduced systemic infection and rapid clearance of the bacterium.

The gene encoding the FTL0552 response-regulator appears present as the first gene of a cluster of five genes. The intergenic space between these genes is either extremely short or absent. Some genes overlap with the putative start codon for truB, located inside the reading frame for rnc and the start codon for rnr is inside the reading frame for truB. Thus, it is unlikely that promoter and transcription termination sequences for each or any of these five genes are located inside this putative operon. RT-PCR analysis revealed that FTL0552 is transcribed as a five-gene operon, which includes lepB, rnc, rnr, and truB. The five-gene arrangement is conserved in Francisella tularensis subsp. holarctica OSU18 (NC008369), Francisella tularensis subsp. tularensis FSC 198 (NC008245), and Francisella tularensis subsp. tularensis SchuS4 (NC006570).

A hallmark of F. tularensis infection is the bacterium's ability to invade and replicate within host macrophages. Once adapted to the host target cells, the bacterium is able to vigorously multiply before the host can offer a protective immune response, and spreads to various organs, such as the liver and spleen. The host-derived response to the rapidly multiplying bacteria results in severe organ damage and is primarily responsible for the high mortality associated with F. tularensis LVS in mice and F. tularensis tularensis (Type A) in humans. The FTL0552 mutant is defective in intracellular replication in macrophages, and is completely avirulent in the mouse model. When boosted, 40% of the BALB/c mice infected with the FTL0552 mutant survived subsequent challenge with the highly virulent SchuS4 strain. Therefore, the FTL0552 mutant is not only highly attenuated in mice, but also retains its antigenic potential and provides partial protection against virulent SchuS4 challenge.

The mutant exhibited reduced dissemination and decreased ability to induce histopathology in the target organ tissues. Mice infected with the FTL0552 mutant were able to clear the bacteria much more efficiently than mice infected with the parental LVS strain. Significantly lower numbers of bacteria disseminated to the spleen and were completely cleared by day 5. After intranasal infection with the mutant strain, the mice were able to completely clear the bacteria from the lung by day 7. The efficiency of clearance correlated with the reduced histopathology evident in the lung, liver, and spleen of FTL0552 mutant infected mice. Although some lesions and inflammation were observed in the liver and lung, there were very few, small lesions in these organs which was dramatically different from time parental LVS strain infected mice. The mutant caused a reduced level of inflammatory cytokine production, yet afforded partial protection to SchuS4 challenge. The low levels of acute phase inflammatory cytokines from the FTL0552 infected lungs correlated with the low level of organ damage seen.

DNA microarray analysis identified 148 genes regulated by FTL0552 in F. tularensis LVS, 75% activated by FTL0552. Among the 113 genes activated by FTL0552 are intracellular growth locus genes found within the FPI, iglA, iglB. iglC and iglD, which are essential for infection and survival within macrophages, type IV pili fiber building block protein, the transport protein ampG, outer membrane protein fopA, and the sodB gene. The loss of expression of these genes is likely to play a role in the impaired intracellular survival and the attenuated virulence in mice observed by the FTL0552 mutant. The attenuation we see in macrophages and in mice can be directly attributed only to the loss of FTL0552.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. The method of inducing an immune response against a γ-proteobacteria comprising:
identifying a target regulatory protein controlling virulence;
constructing a mutation against the target regulatory protein;
incorporating the mutation into the bacterial genome; and
administering the mutated bacteria into an organism;
wherein the mutation modulates protein activity by inhibiting the expression or activity of the target regulatory protein.

2. The method of claim 1, wherein the γ-proteobacteria is F. tularensis.

3. The method of claim 1 wherein the mutation is selected from the group consisting of knockout and deletion mutations.

4. The method of claim 3 wherein the γ-proteobacteria is selected from the group F. tularensis subspecies holartica, F. tularensis subspecies tularensis, and F. tularensis subspecies tularensis SchuS4.

5. The method of claim 1, wherein the target regulatory protein is a homologue of a gene selected from the group consisting of PhoP, PmrA, two component regulatory system genes, TCS response-regulator genes, and transcriptional regulator genes.

6. The method of claim 1, wherein the target regulatory protein controls genes selected from the group consisting of FPI genes, ampC, fopA, sodB, lactamase, permease, oxidative stress survival genes, intracellular survival genes, pili genes, PhoP activated genes, PhoP repressed genes, and pathogenicity island genes.

7. The method of claim 1, wherein the target regulatory protein is selected from the group consisting of FTL 0552 and FTT 1557c.

8. A recombinant attenuated cell comprising a γ-proteobacteria and mutant DNA segment wherein the mutant DNA segment is within a genome sequence encoding a regulatory protein controlling virulence;
the mutant DNA is selected from the group consisting of knockout and deletion mutation; and
wherein the mutation modulates protein activity by inhibiting the expression or activity of the regulatory protein.

9. The cell of claim 8, wherein the genome sequence is a homologue of a gene selected from the group consisting of PhoP, PmrA, two component regulatory system genes, and TCS response-regulator genes.

10. The cell of claim 8, wherein the genome sequence encodes genes selected from the group consisting of FPI genes, ampC, fopA, sodB, lactamase, permease, oxidative stress survival genes, intracellular survival genes, pili genes PhoP activated genes, PhoP repressed genes, and pathogenicity island genes.

11. The cell of claim 8, wherein the γ-proteobacteria is F. tularensis.

12. The cell of claim 11, wherein the mutation is selected from the group consisting of knockout and deletion mutation;
the activity of a regulatory protein is attenuated; and
wherein the regulatory protein is selected from the group consisting of FTL 0552 and FTT 1557c.

13. A mutation of a bacterial gene encoding a regulatory protein that regulates virulence factor genes;
wherein the mutation is selected from the group consisting of knockout and deletion mutation.

14. The mutation of claim 13, wherein the bacterial gene is a homologue of a gene selected from the group consisting of PhoP, PmrA, two component regulatory system genes, TCS response-regulator genes.

15. The mutation of claim 13, wherein the bacterial gene controls genes selected from the group consisting of FPI genes, ampC, fopA, sodB, lactamase, permease, oxidative stress survival genes, intracellular survival genes, pili genes PhoP activated genes, PhoP repressed genes, and pathogenicity island genes.

16. The mutation of claim 13, wherein the bacterial gene is selected from the group consisting of FTL 0552 and FTL 1557c.

17. The mutation of claim 13, wherein the mutation is selected to modulate protein activity by inserting a genetic knockout form of the target protein.

18. The mutation of claim 17, wherein the mutation is selected to inhibit the expression or activity of the target protein.

19. The mutation of claim 13, wherein the mutation is inserted into a bacterial genome.

20. The mutation of claim 19, wherein the bacterial genome belongs to F. tularensis.


SEQUENCE LISTING (18 sequences)

SEQ ID NO: 1 - DNA, 18 bp, Artificial Sequence
  Artificial oligonucleotide based on the pPV vector.  Forms adaptor with P328 to introduce NotI site into pPV1.
tcgacatgcggccgcatt

SEQ ID NO: 2 - DNA, 18 bp, Artificial Sequence
  Artificial oligonucleotide based on the pPV vector.  Forms adaptor with P3278 to introduce NotI site into pPV1.
ctagaatgcggccgcatg

SEQ ID NO: 3 - DNA, 30 bp, Artificial Sequence
  Artificial oligonucleotide based on Francisella tularensis genome.  Forward primer to amplify 5′ flanking region upstream of FTL0552 and insert a NotI restriction enzyme site.
gcgcggccgcgactcattcagtatctggga

SEQ ID NO: 4 - DNA, 45 bp, Artificial Sequence
  Artificial oligonucleotide based on Francisella tularensis genome.  Reverse primer to amplify 5′ flanking region upstream of FTL0552 and insert a NotI restriction enzyme site.
cggcaagaaagccatccagtcatcttcagccaacaatattctcat

SEQ ID NO: 5 - DNA, 46 bp, Artificial Sequence
  Artificial oligonucleotide based on Francisella tularensis genome.  Forward primer to amplify 3′ flanking region downstream of FTL0552.  5′overhang of the primer contains kanR sequences to insert a NotI restriction enzyme site.
gcgggactctggggttcgaaaggtgttggttactttgtacaaaagg

SEQ ID NO: 6 - DNA, 30 bp, Artificial Sequence
  Artificial oligonucleotide based on Francisella tularensis genome.  Reverse primer to amplify 3′ flanking region downstream of FTL0552 and insert a NotI restriction enzyme site.
gcgcggccgctgggactctaaggttttcaa

SEQ ID NO: 7 - DNA, 20 bp, Artificial Sequence
  Artificial oligonucleotide.  Forward primer to amplify amplify the kanR gene from PBBR1MCS2.
actggatggctttcttgccg

SEQ ID NO: 8 - DNA, 21 bp, Artificial Sequence
  Artificial oligonucleotide.  Reverse primer to amplify amplify the kanR gene from PBBR1MCS2.
tttcgaaccccagagtcccgc

SEQ ID NO: 9 - DNA, 23 bp, Artificial Sequence
  Artificial oligonucleotide based on Francisella tularensis genome.  Forward primer to amplify the region within FTL0552 gene to confirm gene knockout and for RT-PCR.
ttcagatggtgaggcagcgcaaa

SEQ ID NO: 10 - DNA, 25 bp, Artificial Sequence
  Artificial oligonucleotide based on Francisella tularensis genome.  Reverse primer to amplify the region within FTL0552 gene to confirm gene knockout and for RT-PCR.
ctttggttaccgtttcagagcttgg

SEQ ID NO: 11 - DNA, 20 bp, Artificial Sequence
  Artificial oligonucleotide based on Francisella tularensis genome.  Forward primer to amplify region outside area of recombination to confirm integration of FTL0552::kan in the chromosome.
cggtgcaaagacaggaaaac

SEQ ID NO: 12 - DNA, 19 bp, Artificial Sequeence
  Artificial oligonucleotide based on Francisella tularensis genome.  Reverse primer to amplify region outside area of recombination to confirm integration of FTL0552::kan in the chromosome.
gtgaccgccctgttcacta

SEQ ID NO: 13 - DNA, 20 bp, Artificial Sequence
  Artificial oligonucleotide based on Francisella tularensis genome.  Amplify gene downstream of FTL0552 for RT-PCR.
gctgatcaggcgagatcttt

SEQ ID NO: 14 - DNA, 20 bp, Artificial Sequence
  Artificial oligonucleotide based on Francisella tularensis genome.  Amplify gene downstream of FTL0552 for RT-PCR.
aattacatcgccaggcaaac

SEQ ID NO: 15 - DNA, 20 bp, Artificial Sequence
  Artificial oligonucleotide based on Francisella tularensis genome.  Amplify gene downstream of FTL0552 for RT-PCR.
accaagctaggcaagaacga

SEQ ID NO: 16 - DNA, 20 bp, Artificial Sequence
  Artificial oligonucleotide based on Francisella tularensis genome.  Amplify gene downstream of FTL0552 for RT-PCR.
tgctaaagctttgggagtca

SEQ ID NO: 17 - DNA, 20 bp, Artificial Sequence
  Artificial oligonucleotide based on Francisella tularensis genome.  House keeping gene for RT-PCR based on mutS gene.
gtggcgtcatcaaagaaggt

SEQ ID NO: 18 - DNA, 20 bp, Artificial Sequence
  Artificial oligonucleotide based on Francisella tularensis genome.  House keeping gene for RT-PCR based on the mutS gene.
cgggctagcgccttttcttt